(19) United States
(12) Patent Application Publication
(10) Pub. No.: US 2009/0187219 A1
(43) Pub. Date: Jul. 23, 2009

(54) Spinal Facet Joint Immobilization Systems and Methods

(76) Inventors: Neal Pachtman, Boca Raton, FL (US); Joseph Mussey, Boca Raton, FL (US)

Correspondence Address: Clements Bernard PLLC, 1901 Roxborough Road, Suite 300, Charlotte, NC, 28211, US

(21) Appl. No.: 12/184,594

(22) Filed: Aug. 1, 2008

Related U.S. Application Data
(60) Provisional application No. 60/953,806, filed on Aug. 3, 2007.

(51) Int. Cl. A61B 17/84; A61F 5/00

(52) U.S. Cl. 606/324; 606/86 A

(57) ABSTRACT

The present invention provides novel systems and methods for aligning and securely holding superior and inferior facets of the spine of a patient to be joined during an interbody fusion or other spinal immobilization procedure (simultaneously compressing the associated facet joint), drilling a hole through which a facet fixation device will be placed, and placing the facet fixation device to a desired penetration depth. The present invention also provides novel facet anchor devices.

### CROSS-REFERENCE TO RELATED APPLICATION

The present non-provisional patent application claims the benefit of priority of U.S. Provisional Patent Application No. 60/953,806, filed on Aug. 3, 2007, and entitled “SPINAL FACET JOINT IMMOBILIZATION SYSTEMS AND METHODS,” the contents of which are incorporated in full by reference herein.

### FIELD OF THE INVENTION

The present invention relates generally to systems and methods for performing spinal and other surgical procedures. More specifically, the present invention relates to systems and methods for immobilizing a facet joint of the spine of a patient or other similar joint or bony structure.

### BACKGROUND OF THE INVENTION

When confronted with various spinal diseases and injuries, it is often desirable for a spinal surgeon to perform an interbody fusion or the like, whereby adjacent vertebrae are fused together using a bone graft and/or an implantable device, or otherwise immobilize a portion of the spine of a patient. Typically, in the interbody fusion case, the adjacent vertebrae are immobilized while the bone graft is allowed to “take,” for example, using a conventional pedicle screw system, a plate system, or the like. Such a pedicle screw system consists of a plurality of pedicle screws that are anchored to adjacent levels of the spine and connected with stabilizing rods or the like. Such a plate system consists of a plate that is anchored to adjacent levels of the spine and, optionally, connected to the implantable device.

Another attractive option when treating various spinal diseases and injuries is to immobilize the associated facet joint(s) using one or more facet bolts or the like. In order to accomplish this, the superior and inferior facets to be joined must be aligned and securely held during drilling and bolt placement, for example. It is also desirable that they are compressed either before or during drilling and bolt placement. This can be a tricky process, which is never desirable during a surgical procedure.

Thus, what are needed in the art are simplified systems and methods for aligning and securely holding superior and inferior facets to be joined (simultaneously compressing the associated facet joint), drilling the hole through which a facet fixation device will be placed, and placing the facet fixation device to a desired penetration depth. Such systems and methods are provided by the present invention.

### BRIEF SUMMARY OF THE INVENTION

In various exemplary embodiments, the present invention provides novel systems and methods for aligning and securely holding superior and inferior facets of the spine of a patient to be joined during an interbody fusion or other spinal immobilization procedure (simultaneously compressing the associated facet joint), drilling a hole through which a facet fixation device will be placed, and placing the facet fixation device to a desired penetration depth. In various exemplary embodiments, the present invention also provides novel facet anchor devices.

In one exemplary embodiment, the present invention provides a facet clamp device, including: a pair of elongate arms joined at a pivot joint; a pair of washers coupled to the pair of elongate arms, wherein the pair of washers are configured to engage a pair of facets associated with a spine of a patient; and a compression lock coupled to the pair of elongate arms, wherein the compression lock is operable for holding the pair of elongate arms secure with respect to one another. The facet clamp device also includes a pair of handles coupled to the pair of elongate arms opposite the pair of washers, wherein the pair of handles are disposed at an angle of approximately 90 degrees to the pair of elongate arms. Optionally, each washer of the pair of washers comprises a friction surface for engaging one of the pair of facets. Optionally, one or more washers of the pair of washers are pivotable with respect to the associated elongate arm of the pair of elongate arms. Optionally, one or more washers of the pair of washers define a bore through which one or more of a drill bit, a depth gauge, and a facet fixation device is/are selectively placed.

In another exemplary embodiment, the present invention provides a facet anchor device, including: an elongate locking member configured to be selectively disposed through and at least partially within a bore formed through a pair of facets associated with a spine of a patient, the elongate locking member including one or more inwardly biased locking structures, the one or more inwardly biased locking structures each including a lip structure; and a threaded plug member configured to be selectively disposed within a threaded bore formed through the elongate locking member, thereby outwardly biasing the inwardly biased locking structures and lip structures, the lip structures preventing the elongate locking member from being removed from the bore formed through the pair of facets. The elongate locking member also includes a head nut coupled to the elongate locking member opposite the lip structures. Optionally, an outside surface of the elongate locking member and an inside surface of the bore formed through the pair of facets are correspondingly threaded. Optionally, the elongate locking member and the one or more inwardly biased locking structures are integrally formed. Optionally, the inwardly biased locking structures and the lip structures are integrally formed.

In a further exemplary embodiment, the present invention provides a facet bolt implantation device, including. a substantially arcuate bridge member; a pair of washers coupled to the substantially arcuate bridge member, wherein the pair of washers are configured to engage a pair of facets associated with a spine of a patient; and a cannulated compression mechanism coupled to the substantially arcuate bridge member, wherein the cannulated compression mechanism is configured to selectively advance one washer of the pair of washers towards the other washer of the pair of washers, and wherein the cannulated compression mechanism is configured to receive a facet fixation device there through. Optionally, each washer of the pair of washers includes a friction surface for engaging one of the pair of facets. Optionally, one or more washers of the pair of washers are pivotable with respect to the substantially arcuate bridge member. Optionally, the substantially arcuate bridge member and the cannulated compression mechanism are each manufactured from a radiolucent material. The facet bolt implantation device also includes a depth gauge associated with the cannulated compression mechanism, wherein the depth gauge is operable for indicating a deflection of the pair of washers from an initial position to a subsequent position, this deflection indicating what size facet fixation device should be used.

### BRIEF DESCRIPTION OF THE DRAWINGS

The present invention is illustrated and described herein with reference to the various drawings, in which like reference numbers are used to denote like system components and/or method steps, as appropriate, and in which:

FIG. 1 is a planar side view (as well as a partial planar front view) illustrating one exemplary embodiment of a facet clamp device of the present invention;

FIG. 2 is a cross-sectional side view illustrating one exemplary embodiment of a facet anchor device of the present invention in a partially deployed state;

FIG. 3 is a cross-sectional side view illustrating one exemplary embodiment of the facet anchor device of the present invention in a fully deployed state;

FIG. 4 is an exploded planar side view illustrating one exemplary embodiment of a facet bolt implantation device of the present invention; and

FIG. 5 is an exploded planar side view illustrating one exemplary embodiment of a multi-purpose ratchet device of the present invention.

### DETAILED DESCRIPTION OF THE INVENTION

In general, the present invention provides systems and methods for immobilizing a facet joint of the spine of a patient or other similar joint or bony structure.

In various exemplary embodiments, the present invention provides novel systems and methods for aligning and securely holding superior and inferior facets of the spine of a patient to be joined during an interbody fusion or other spinal immobilization procedure (simultaneously compressing the associated facet joint), drilling a hole through which a facet fixation device will be placed, and placing the facet fixation device to a desired penetration depth. In various exemplary embodiments, the present invention also provides novel facet anchor devices.

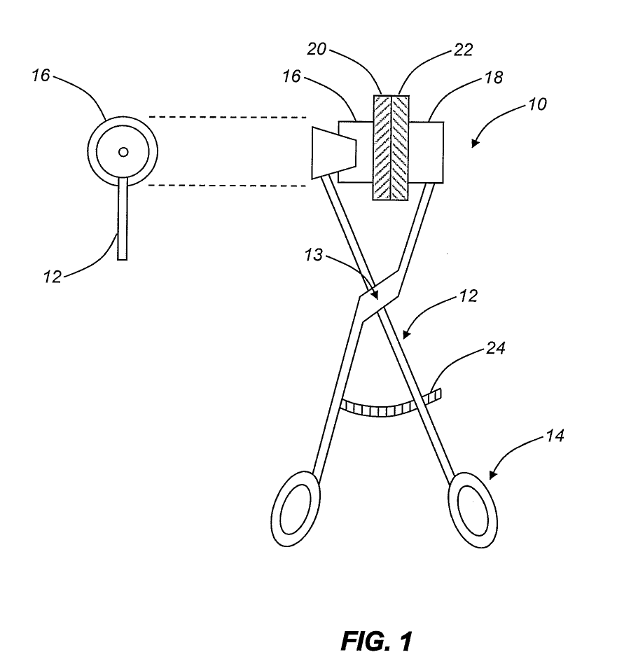

Referring to FIG. 1, in one exemplary embodiment, the facet clamp device 10 of the present invention includes a pair of elongate arms 12 having a pair of handles 14 or the like for actuating the elongate arms 12 about a centrally disposed pivot joint 13. In operation, the pair of elongate arms 12 “scissor” with respect to one another. It will be readily apparent to those of ordinary skill in the art, however, that another actuation mechanism relative to the movement of the pair of elongate arms 12 could be utilized. A proximal washer 16 is attached to one of the elongate arms 12 and a lock washer 18 is attached to another of the elongate arms 12. Optionally, one or both of the proximal washer 16 and the lock washer 18 may pivot with respect to the attached elongate arm 12. Collectively, the proximal washer 16 and the lock washer 18 are operable for selectively engaging, compressing, and securely holding a superior facet 20 and an inferior facet 22 of the spine of a patient or other similar joint or bony structures. Accordingly, the engaging face of the proximal washer 16 and the lock washer 18 may consist of a slip-resistant material, or incorporate one or more protruding “biting” friction structures, suitable for securely holding the superior facet 20 and the inferior facet 22. Likewise, the elongate arms 12 incorporate a compression lock 24 or the like. Thus, once the facet clamp device 10 has been positioned and placed, it becomes an essentially “hands free” device. Optionally, the handles 14 of the facet clamp device 10 are bent approximately 90 degrees relative to the elongate arms 12, such that the device may be conveniently positioned and placed around the facets 20 and 22.

Once positioned and placed around the facets 20 and 22, the proximal washer 16 may act as a drill guide and the compression lock 24 may act as a depth gauge for the subsequent placement of a facet fixation device (not illustrated) through the proximal washer 16. The facet clamp device 10 may be made of any surgically-compatible material.

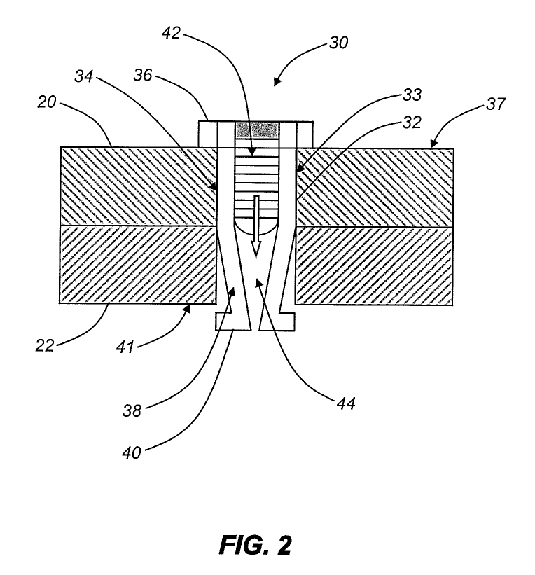

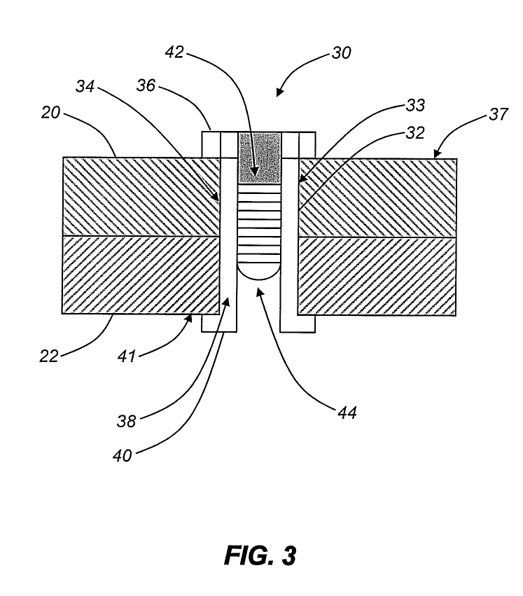

Referring to FIGS. 2 and 3, in one exemplary embodiment, the facet anchor device 30 of the present invention includes a substantially hollow (e.g. cylindrical) elongate locking member 32 that is selectively disposed through a bore 34 that is drilled through the superior facet 20 and the inferior facet 22 of interest. The elongate locking member 32 has an inner diameter and an outer diameter, the later of which substantially corresponds to an inner diameter of the bore 34. Optionally, the exterior surface 33 of the elongate locking member 32 is threaded, such that a driver (not illustrated) is required to advance the elongate locking member 32 into and/or back the elongate locking member 32 out of the bore 34, which may also be threaded. The elongate locking member 32 also includes a head nut 36 that is configured to sit substantially adjacent to and/or flush with the surface 37 of the superior facet 20. The elongate locking member 32 further includes one or more inwardly biased locking portions 38 each having a lip structure 40 that is configured to selectively engage the surface 41 of the inferior facet 22. For example, the lower portion of the elongate locking member 32 may be cut or segmented to form “petals” or the like that serve as the inwardly biased locking portions 38. The lower portion of each of these “petals” or the like may then be bent outwards to form the lip structures 40. In operation, the “petals” act as “spring loaded” locking tabs.

These inwardly biased locking portions 38 are biased outwards by driving a threaded plug 42 through a threaded bore 44 manufactured through the elongate locking member 32, thereby causing the lip structures 40 to engage the surface 41 of the inferior facet 22. The facet anchor device 30 may be made of any surgically-compatible material.

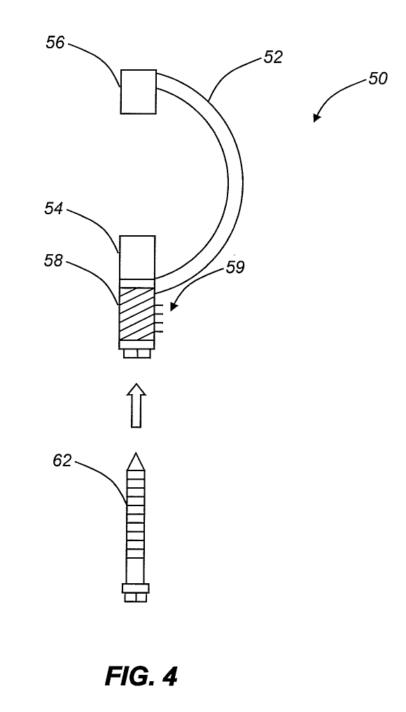

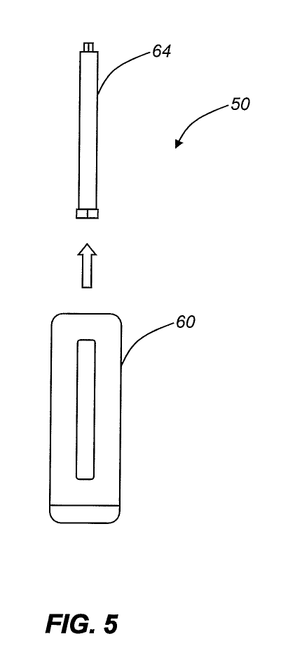

Referring to FIGS. 4 and 5, in one exemplary embodiment, the facet bolt implantation device 50 of the present invention includes a substantially arcuate bridge member 52 or the like, a proximal washer 54 coupled to the bridge member 52, and a lock washer 56 coupled to the bridge member 52. Optionally, one or both of the proximal washer 54 and the lock washer 56 may pivot with respect to the bridge member 52. Collectively, the proximal washer 54 and the lock washer 56 are operable for selectively engaging, compressing, and securely holding a superior facet (not illustrated) and an inferior facet (not illustrated) of the spine of a patient or other similar joint or bony structures. As described above, the engaging face of the proximal washer 54 and the lock washer 56 may consist of a slip-resistant material, or incorporate one or more protruding “biting” friction structures, suitable for securely holding the superior and inferior facets of interest.

In operation, the proximal washer 54 is advanced towards the lock washer 56 using a cannulated compressor mechanism 58. This cannulated compressor mechanism 58 has external threading corresponding to internal threading of a portion of the bridge member 52. Thus, the cannulated compressor mechanism 58 is driven through the bridge member 52 using a driver handle 60 (FIG. 5) or the like. Optionally, the bridge member 52 and the cannulated compressor mechanism 58 are manufactured from a radiolucent material, and the cannulated compressor mechanism 58 includes scale markings 59 indicating the degree of advancement of the proximal washer 54 towards the lock washer 56, thereby indicating the length of bolt that should be used subsequently.

The bolt 62 is inserted through the cannulated compressor mechanism 58 and is driven into the superior and inferior facets of interest, optionally subsequent to a drilling step, using the driver handle 60 and a driver extension shaft 64 (FIG. 5). As a safety precaution, the driver handle 60 may have a break-away feature at 25 in/lbs, for example. Advantageously, the facet bolt implantation device 50 of the present invention allows both facets to be compressed before bolt fixation.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the invention, are contemplated thereby, and are intended to be covered by the following claims.

What is claimed is:

1. A facet clamp device, comprising:
a pair of elongate arms joined at a pivot joint;
a pair of washers coupled to the pair of elongate arms, wherein the pair of washers are configured to engage a pair of facets associated with a spine of a patient; and
a compression lock coupled to the pair of elongate arms, wherein the compression lock is operable for holding the pair of elongate arms secure with respect to one another.

2. The facet clamp device of claim 1, further comprising a pair of handles coupled to the pair of elongate arms opposite the pair of washers, wherein the pair of handles are disposed at an angle of approximately 90 degrees to the pair of elongate arms.

3. The facet clamp device of claim 1, wherein each washer of the pair of washers comprises a friction surface for engaging one of the pair of facets.

4. The facet clamp device of claim 1, wherein one or more washers of the pair of washers are pivotable with respect to the associated elongate arm of the pair of elongate arms.

5. The facet clamp device of claim 1, wherein one or more washers of the pair of washers define a bore through which one or more of a drill bit, a depth gauge, and a facet fixation device is/are selectively placed.

6. A facet anchor device, comprising:
an elongate locking member configured to be selectively disposed through and at least partially within a bore formed through a pair of facets associated with a spine of a patient, the elongate locking member comprising one or more inwardly biased locking structures, the one or more inwardly biased locking structures each comprising a lip structure; and
a threaded plug member configured to be selectively disposed within a threaded bore formed through the elongate locking member, thereby outwardly biasing the inwardly biased locking structures and lip structures, the lip structures preventing the elongate locking member from being removed from the bore formed through the pair of facets.

7. The facet anchor device of claim 6, the elongate locking member further comprising a head nut coupled to the elongate locking member opposite the lip structures.

8. The facet anchor device of claim 6, wherein an outside surface of the elongate locking member and an inside surface of the bore formed through the pair of facets are correspondingly threaded.

9. The facet anchor device of claim 6, wherein the elongate locking member and the one or more inwardly biased locking structures are integrally formed.

10. The facet anchor device of claim 6, wherein the inwardly biased locking structures and the lip structures are integrally formed.

11. A facet bolt implantation device, comprising:
a substantially arcuate bridge member;
a pair of washers coupled to the substantially arcuate bridge member, wherein the pair of washers are configured to engage a pair of facets associated with a spine of a patient; and
a cannulated compression mechanism coupled to the substantially arcuate bridge member, wherein the cannulated compression mechanism is configured to selectively advance one washer of the pair of washers towards the other washer of the pair of washers, and wherein the cannulated compression mechanism is configured to receive a facet fixation device there through.

12. The facet bolt implantation device of claim 11, wherein each washer of the pair of washers comprises a friction surface for engaging one of the pair of facets.

13. The facet bolt implantation device of claim 11, wherein one or more washers of the pair of washers are pivotable with respect to the substantially arcuate bridge member.

14. The facet bolt implantation device of claim 11, wherein the substantially arcuate bridge member and the cannulated compression mechanism are each manufactured from a radiolucent material.

15. The facet bolt implantation device of claim 11, further comprising a depth gauge associated with the cannulated compression mechanism, wherein the depth gauge is operable for indicating a deflection of the pair of washers from an initial position to a subsequent position, this deflection indicating what size facet fixation device should be used.
